(19) United States
(12) Patent Application Publication
(10) Pub. No.: US 2016/0128954 A1
(43) Pub. Date: May 12, 2016

(54) Methods of Treating Huntington's Disease Using Cysteamine Compositions

(71) Applicant: Raptor Pharmaceuticals Inc., Novato, CA (US)

(72) Inventor: Patrice Rioux, San Jose, CA (US)

(21) Appl. No.: 14/933,529

(22) Filed: Nov. 5, 2015

Related U.S. Application Data
(60) Provisional application No. 62/075,536, filed on Nov. 5, 2014.

(51) Int. Cl. A61K 31/145; A61K 45/06

(52) U.S. Cl. CPC A61K 31/145; A61K 45/06

(57) ABSTRACT

The present disclosure relates in general to methods for the treatment of neurodegenerative disease, such as Huntington's Disease, using compositions comprising cysteamine or cystamine or salts or derivatives thereof.

This application claims the priority benefit of U.S. Provisional Patent Application No. 62/075,536, filed Nov. 5, 2014, herein incorporated by reference.

### FIELD OF THE INVENTION

The present disclosure relates in general to methods for the treatment of neurodegenerative disease, such as Huntington's Disease, using compositions comprising cysteamine or cystamine or salts or derivatives thereof.

### BACKGROUND OF THE INVENTION

Huntington's disease (HD) is an adult-onset neurodegenerative disorder for which treatment strategies have helped address certain symptoms of HD, but remain ineffective at truly treating the disease. HD is an autosomal dominant genetic disorder with a prevalence of about 5-10 per 100,000 in the Caucasian population. Clinical symptoms include chorea and behavioral disorders but the most problematic features of the disease are slowly progressive motor dysfunction and impaired cognition (1). The pathology of HD is characterized by the presence of neuritic and intranuclear inclusions in neurons and relatively selective neural loss in the striatum and the deeper layers of the cerebral cortex. HD is caused by a Cytosine-Adenine-Guanine (CAG) triplet repeat expansion in the first exon of the HTT gene leading to an expanded polyglutamine stretch in the huntingtin protein (2). HD develops when the polyglutamine expansion exceeds 35 CAG, a point that enlarges the polyglutamine stretch past a critical threshold that predisposes to aggregation. There is an inverse correlation between the number of CAG and the age at onset (3). Mutant huntingtin has been implicated in the disruption of many cellular processes, including protein clearance, protein-protein interaction, mitochondrial function, axonal trafficking, N-methyl-D-aspartate receptor activation, gene transcription and post-translational modification (4,5). Although mutant huntingtin has a widespread distribution in neuronal and non-neuronal tissues, the medium spiny GABAergic neurons of the striatum exhibit the most pronounced vulnerability (5).

Despite progress in the understanding of the pathogenesis of HD, neuroprotective or curative strategies remain ineffective, and the average lifespan is 10 to 20 years after disease onset (6). Tetrabenazine is the only drug licensed in North America and some European countries to treat HD, and treats chorea associated with HD but does not improve cognition, slow decline in motor function, or show benefit on functional scales (7). Patients often are prescribed antipsychotics and/or antidepressants to treat behavior or mood disorders, but there is no evidence that they improve motor function or alter disease progression.

### SUMMARY OF THE INVENTION

The present invention relates to treatment of a neurodegenerative disease, such as Huntington's Disease, using a composition comprising a cysteamine product formulated for administration less than four times daily, e.g., twice daily. It was discovered herein that administration of a cysteamine composition is effective to improve motor function of patients with HD.

In various embodiments, the disclosure provides a method for treating Huntington's Disease in a patient comprising administering cysteamine or a pharmaceutically acceptable salt thereof or cystamine or a pharmaceutically acceptable salt thereof twice daily at a total daily dose of 1000 to 1500 mg per day. In various embodiments, the cysteamine or a pharmaceutically acceptable salt thereof or cystamine or a pharmaceutically acceptable salt thereof is administered in a total daily dose of approximately 1200 mg given in two doses. In various embodiments, the administration is given in two daily doses of approximately 600 mg each.

In various embodiments, the cysteamine or a pharmaceutically acceptable salt thereof or cystamine or a pharmaceutically acceptable salt thereof is administered in a total daily dose of approximately 1000, 1100, 1200, 1300, 1400 or 1500 mg per day in one, two or three doses.

In various embodiments, the cysteamine or a pharmaceutically acceptable salt thereof or cystamine or a pharmaceutically acceptable salt thereof is administered in a total daily dose of 15 to 25 mg/kg, 15 to 20 mg/kg or 10 to 20 mg/kg, over one, two or three doses daily.

In various embodiments, the cysteamine or a pharmaceutically acceptable salt thereof or cystamine or a pharmaceutically acceptable salt thereof is in a delayed release or extended release formulation. In various embodiments, the delayed release composition is enterically coated. For example, the coating can be selected from the group consisting of polymerized gelatin, shellac, methacrylic acid copolymer type CNF, cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethylcellulose (CMEC), hydroxypropyl methylcellulose acetate succinate (HPMCAS), and acrylic acid polymers and copolymers, typically formed from methyl acrylate, ethyl acrylate, methyl methacrylate and/or ethyl methacrylate with copolymers of acrylic and methacrylic acid esters. The composition can be administered orally or parenterally. Additional enteric coatings and formulations contemplated herein are discussed further in the Detailed Description.

In some embodiments, the delayed release formulation comprises an enteric coating that releases the cysteamine or cystamine when the formulation reaches the small intestine or a region of the gastrointestinal tract of a subject in which the pH is greater than about pH 4.5. In various embodiments, the formulation releases at a pH of about 4.5 to 6.5, 4.5 to 5.5, 5.5 to 6.5 or about pH 4.5, 5.0, 5.5, 6.0 or 6.5.

In various embodiments, the cysteamine, cystamine or pharmaceutically acceptable salt thereof is formulated in a tablet or capsule which is enterically coated.

In various embodiments, the cysteamine or a pharmaceutically acceptable salt thereof or cystamine or a pharmaceutically acceptable salt thereof product further comprises a pharmaceutically acceptable carrier. It is further contemplated that the cysteamine product is formulated as a sterile pharmaceutical composition.

In various embodiments, the administration results in a slower progression in decline of total motor score compared to a subject not receiving cysteamine or a pharmaceutically acceptable salt thereof or cystamine or a pharmaceutically acceptable salt thereof. In some embodiments, the slower progression is a result in a decreased change in one or more motor scores selected from the group consisting of chorea subscore, balance and gait subscore, hand movements subscore, eye movement subscore and maximal dystonia subscore.

In certain embodiments, alteration in one or more symptoms in patients receiving cysteamine or a pharmaceutically acceptable salt thereof or cystamine or a pharmaceutically acceptable salt thereof is shown to be beneficial by at least 10%, 20%, 25%, 30%, 35%, 40%, 45%, 50%, 55%, 60%, 65%, 70%, 75% or more compared to baseline assessment of the symptom. In certain embodiments, the rate of progression or decline in total motor score is slowed, by at least 20%, 25%, 30%, 35%, 40%, 45%, 50%, 55%, 60%, 65%, 70%, 75% or more. Measurement may be performed using the Unified Huntington Disease Rating Scale (UHDRS).

Additional indicia of a slower decline in symptoms of HD are measured using change from baseline in one or more of the following parameters: using standardized tests for (i) functional assessment (UHDRS Total Functional Capacity, Independence Scale); (ii) neuropsychological assessment (UHDRS Cognitive Assessment, Mattis Dementia Rating Scale, Trail Making Test A and B, Figure Cancellation Test, Hopkins Verbal Learning Test, Articulation Speed Test); and (iii) psychiatric assessment (UHDRS Behavioral Assessment, Montgomery and Asberg Depression Rating Scale).

In certain embodiments, the symptoms are assayed at 6 months, 12 months, 18 months or 2 years or more after administration.

The disclosure also provides a method for slowing the progression of brain and striatal atrophies in a subject suffering from a neurodegenerative disease comprising administering to a subject in need thereof a composition comprising cysteamine or a pharmaceutically acceptable salt thereof or cystamine or a pharmaceutically acceptable salt thereof in a total daily dose of approximately 1000 to 1500 mg, or approximately 1000, 1100, 1200, 1300, 1400 or 1500 mg, given in two doses.

In various embodiments, the disclosure contemplates a method for treating dystonia in a subject suffering from a neurodegenerative disease comprising administering to a subject in need thereof a composition comprising cysteamine or a pharmaceutically acceptable salt thereof or cystamine or a pharmaceutically acceptable salt thereof in a total daily dose of approximately 1000 to 1500 mg, or approximately 1000, 1100, 1200, 1300, 1400 or 1500 mg, given in two doses.

Also contemplated is a method for decreasing levels of transglutaminase in a subject suffering from a neurodegenerative disease comprising administering to a subject in need thereof a composition comprising cysteamine or a pharmaceutically acceptable salt thereof or cystamine or a pharmaceutically acceptable salt thereof in a total daily dose of approximately 1000 to 1500 mg, or approximately 1000, 1100, 1200, 1300, 1400 or 1500 mg, given in two doses. In various embodiments, the transglutaminase is Tgase 2.

In various embodiments, the subject suffering from a neurodegenerative disease suffers from Huntington's disease. In various embodiments, it is contemplated that the cysteamine or cystamine or pharmaceutically acceptable salt thereof is useful to treat any stage of Huntington's disease (stages 1-5), including early stages, such as stage 1 or stage 2, intermediate stages, such as stage 3 and stage 4, and advanced Huntington's Disease, such as stage 5 HD. Further discussion of the stages of HD are provided in the Detailed Description.

For any of the methods or uses herein, the cysteamine or a pharmaceutically acceptable salt thereof or cystamine or a pharmaceutically acceptable salt thereof is administered in a total daily dose of approximately 1200 mg given in two doses. In various embodiments, the administration is given in two daily doses of approximately 600 mg each.

It is contemplated that there may be a certain period during treatment where the dose of cysteamine or a pharmaceutically acceptable salt thereof or cystamine or a pharmaceutically acceptable salt thereof needs to be varied during a ramp up or ramp down phase.

In various embodiments, the total daily dose of cysteamine or a pharmaceutically acceptable salt thereof or cystamine or a pharmaceutically acceptable salt thereof is between 500 to 2000 mg, 750 to 1750 mg, 1000 to 1500 mg, or may range between any two of the foregoing values. In various embodiments, the total daily dose of cysteamine or a pharmaceutically acceptable salt thereof or cystamine or a pharmaceutically acceptable salt thereof is 500, 600, 700, 800, 900, 1000, 1100, 1200, 1300, 1400, 1500, 1600, 1700, 1800, 1900 or 2000 mg per day. It is contemplated that any of the foregoing doses is administered twice daily. It is further contemplated that any of the foregoing doses is administered in two equal doses daily.

In various embodiments of the disclosure, the cysteamine or a pharmaceutically acceptable salt thereof or cystamine or a pharmaceutically acceptable salt thereof is administered at a daily dose ranging from about 10 mg/kg to about 250 mg/kg, or from about 100 mg/kg to about 250 mg/kg, or from about 60 mg/kg to about 100 mg/kg or from about 50 mg/kg to about 90 mg/kg, or from about 30 mg/kg to about 80 mg/kg, or from about 20 mg/kg to about 60 mg/kg, or from about 10 mg/kg to about 50 mg/kg. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 225 mg/kg, 250 mg/kg, 275 mg/kg, 300 mg/kg, 325 mg/kg, 350 mg/kg, 375 mg/kg, 400 mg/kg, 425 mg/kg, 450 mg/kg, 475 mg/kg, 500 mg/kg, 525 mg/kg, 550 mg/kg, 575 mg/kg, 600 mg/kg, 625 mg/kg, 650 mg/kg, 675 mg/kg, 700 mg/kg, 725 mg/kg, 750 mg/kg, 775 mg/kg, 800 mg/kg, 825 mg/kg, 850 mg/kg, 875 mg/kg, 900 mg/kg, 925 mg/kg, 950 mg/kg, 975 mg/kg or 1000 mg/kg, or may range between any two of the foregoing values. In some embodiments, the cysteamine product is administered at a total daily dose of from approximately 0.25 g/m² to 4.0 g/m² body surface area, about 0.5-2.0 g/m² body surface area, or 1-1.5 g/m² body surface area, or 1-1.95g/m² body surface area, or 0.5-1 g/m² body surface area, or about 0.7-0.8 g/m² body surface area, or about 1.35 g/m² body surface area, or about 1.3 to about 1.95 grams/m²/day, or about 0.5 to about 1.5 grams/m2/day, or about 0.5 to about 1.0 grams/m²/day, e.g., at least about 0.3, 0.4, 0.5, 0.6, 0.7, 0.8, 0.9, 1.0, 1.1, 1.2, 1.3, 1.4, 1.5, 1.6, 1.7, 1.8, 1.9 or 2 g/m², or up to about 0.8, 0.9, 1.0, 1.1, 1.2, 1.3, 1.4, 1.5, 1.6, 1.7, 1.8, 1.9, 2.0, 2.2, 2.5, 2.7, 3.0, 3.25, 3.5 or 3.75 g/m² or may range between any two of the foregoing values.

Aspects of the invention that are described herein as methods (especially methods that involve treatment) can alternatively be described as (medical) uses of cysteamine or a pharmaceutically acceptable salt thereof or cystamine or a pharmaceutically acceptable salt thereof. For example, in one variation, described herein the use of cysteamine or a pharmaceutically acceptable salt thereof or cystamine or a pharmaceutically acceptable salt thereof to treat Huntington's Disease. In another variation, described herein is a composition comprising cysteamine or a pharmaceutically acceptable salt thereof or cystamine or a pharmaceutically acceptable salt thereof for use in the treatment of Huntington's Disease, wherein the composition is administered in a total daily dose of approximately 1000 to 1500 mg given in two doses.

The agents and compositions described herein for use in treatment are themselves aspects of the invention also, e.g., as compositions of matter.

In the treatment methods (or uses) described herein, the methods optionally comprise administering an adjunct therapy to the subject in combination with the cysteamine, cystamine or pharmaceutically acceptable salts thereof. In some embodiments, the adjunct therapy is selected from the group consisting of antipsychotics, antidepressants, vesicular monoamine transporter (VMAT)-inhibitors such as tetrabenazine, dopamine inhibitors, laquinimod, CNS-immunomodulators, neuroprotective factors, BDNF and agents that upregulate BDNF, ampakines, positive modulators of AMPA-type glutamate receptors, activators of BDNF receptor TrkB and gene therapy.

Antidepressants include: SSRI antidepressants, such as fluoxetine, citalopram and paroxetine, tricyclic antidepressants, such as amitriptyline, other types of antidepressants, including mirtazapine, duloxetine and venlafaxine.

Antipsychotic medication includes risperidone, olanzapine, aripiprazole, tiapride and quetiapine, benzodiazepines, such as clonazepam and diazepam, and mood stabilizers, such as carbamazepine.

In some embodiments, the methods (or uses) described herein further comprise administering a further therapeutic agent selected from the group consisting of tetrabenazine, laquinimod, BDNF, ampakines, fluoxetine, citalopram, paroxetine, amitriptyline, mirtazapine, duloxetine, venlafaxine, risperidone, olanzapine, aripiprazole, tiapride, quetiapine, clonazepam diazepam and carbamazepine.

In various embodiments, the subject is not concurrently taking tetrabenazine.

In various embodiments, the cysteamine or a pharmaceutically acceptable salt thereof or cystamine or a pharmaceutically acceptable salt thereof is administered parenterally or orally. In various embodiments, the cysteamine or a pharmaceutically acceptable salt thereof or cystamine or a pharmaceutically acceptable salt thereof further comprises a pharmaceutically acceptable carrier. It is further contemplated that the cysteamine or a pharmaceutically acceptable salt thereof or cystamine or a pharmaceutically acceptable salt thereof is formulated as a sterile pharmaceutical composition.

In various embodiments, the methods herein comprise administering cysteamine or a pharmaceutically acceptable salt thereof. In some embodiments, the salt is cysteamine bitartrate or cysteamine hydrochloride. In various embodiments, the cysteamine birtarte or cysteamine hydrochloride is in a delayed release formulation.

With respect to any combination treatments described herein, the cysteamine or a pharmaceutically acceptable salt thereof or cystamine or a pharmaceutically acceptable salt thereof can be administered simultaneously with the other active agents, which may be in admixture with the agent or may be in a separate composition. Each composition preferably includes a pharmaceutically acceptable diluent, adjuvant, or carrier. When the agents are separately administered, they may be administered in any order.

In another aspect, described herein is a method of increasing levels of brain derived neurotrophic factor (BDNF) activity in a brain or neuronal cell comprising contacting the cell with cysteamine, cystamine or pharmaceutically acceptable salt thereof in an amount effective to increase BDNF activity in the cell. In some embodiments, increased levels of BDNF is demonstrated when compared to levels before administration of the cysteamine or a pharmaceutically acceptable salt thereof or cystamine or a pharmaceutically acceptable salt thereof.

It is also contemplated that cysteamine has other effects in vivo in HD patients, including but not limited to inhibition of transglutaminase 2 (TG2) activity, inhibition of the pro-apoptotic activity of caspase 3, increasing the production of heat shock proteins, which prevents protein misfolding and assists the refolding of misfolded proteins, increasing the level of antioxidants such as glutathione, increasing the release of BDNF, which can promote striatal neuron survival, increasing levels of the heat shock DnaJ-containing protein 1b (HSJ1b), and increasing the level of cysteine in the brain.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to paragraph subject matter described in the prior art work of others. Therefore, in the event that statutory prior art within the scope of a paragraph is brought to the attention of the applicant(s) by a Patent Office or other entity or individual, the applicant(s) reserve the right to exercise amendment rights under applicable patent laws to redefine the subject matter of such a paragraph to specifically exclude such statutory prior art or obvious variations of statutory prior art from the scope of such a paragraph. Variations of the invention defined by such amended paragraphs also are intended as aspects of the invention.

### BRIEF DESCRIPTION OF THE DRAWINGS

FIG. 1A shows the mean plot change in UHDRS TMS by visit in intent-to-treat population. Mean changes from baseline±standard error from repeated-measures mixed-effect model with baseline, center, CAG repeats, age and BMI as covariates. FIG. 1B shows the mean plot change in UHDRS TMS by visit in the per-protocol population of patients not taking tetrabenazine (NoTBZ). Mean change from baseline±standard error from repeated measures mixed effect model with baseline, center, CAG repeats, age and BMI as covariates.

FIG. 2 is a Forest plot for UHDRS TMS and subscores in intent-to-treat population and the per-protocol population of patients not taking tetrabenazine (NoTBZ). General linear mixed model repeatedly measured overtime with baseline, center, CAG repeats, age and BMI as covariates. All endpoints are standardized with their baseline values to be represented on the same scale. Dotted line shows no effect point and bold line shows primary endpoint treatment effect.

### DETAILED DESCRIPTION

The present disclosure relates to the treatment of neurodegenerative disease, such as Huntington's Disease, using cysteamine or a pharmaceutically acceptable salt thereof or cystamine or a pharmaceutically acceptable salt thereof formulated for administration, for example, twice daily.

### Definitions

As used herein and in the appended claims, the singular forms “a,” “and,” and “the” include plural referents unless the context clearly dictates otherwise. Thus, for example, reference to “a derivative” includes a plurality of such derivatives and reference to “a patient” includes reference to one or more patients and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and products, the exemplary methods, devices and materials are described herein.

The documents discussed above and throughout the text are provided solely for their disclosure prior to the filing date of the present application. Nothing herein is to be construed as an admission that the inventors are not entitled to antedate such disclosure by virtue of prior disclosure. Each document is incorporated by reference in its entirety with particular attention to the disclosure for which it is cited.

The following references provide one of skill with a general definition of many of the terms used in this disclosure: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

As used herein, a “therapeutically effective amount” or “effective amount” refers to that amount of a cysteamine product, e.g., cysteamine or a pharmaceutically acceptable salt thereof or cystamine or a pharmaceutically acceptable salt thereof, sufficient to result in amelioration of symptoms, for example, treatment, healing, prevention or amelioration of the relevant medical condition, or an increase in rate of treatment, healing, prevention or amelioration of such conditions, typically providing a statistically significant improvement in the treated patient population. When referencing an individual active ingredient, administered alone, a therapeutically effective dose refers to that ingredient alone. When referring to a combination, a therapeutically effective dose refers to combined amounts of the active ingredients that result in the therapeutic effect, whether administered in combination, including serially or simultaneously. In various embodiments, a therapeutically effective amount of the cysteamine product ameliorates symptoms associated with various neurodegenerative diseases, including but not limited to, bradykinesia, dystonia, motor deficiencies, cognitive dysfunction, and psychiatric episodes, including depression.

“Treatment” refers to prophylactic treatment or therapeutic treatment. In certain embodiments, “treatment” refers to administration of a compound or composition to a subject for therapeutic or prophylactic purposes.

A “therapeutic” treatment is a treatment administered to a subject who exhibits signs or symptoms of pathology for the purpose of diminishing or eliminating those signs or symptoms. The signs or symptoms may be biochemical, cellular, histological, functional or physical, subjective or objective.

A “prophylactic” treatment is a treatment administered to a subject who does not exhibit signs of a disease or exhibits only early signs of the disease, for the purpose of decreasing the risk of developing pathology. The compounds or compositions of the disclosure may be given as a prophylactic treatment to reduce the likelihood of developing a pathology or to minimize the severity of the pathology, if developed.

“Diagnostic” means identifying the presence, extent and/or nature of a pathologic condition. Diagnostic methods differ in their specificity and selectivity. While a particular diagnostic method may not provide a definitive diagnosis of a condition, it suffices if the method provides a positive indication that aids in diagnosis.

“Pharmaceutical composition” refers to a composition suitable for pharmaceutical use in a subject animal, including humans and mammals. A pharmaceutical composition comprises a therapeutically effective amount of a cysteamine product, optionally another biologically active agent, and optionally a pharmaceutically acceptable excipient, carrier or diluent. In an embodiment, a pharmaceutical composition encompasses a composition comprising the active ingredient(s), and the inert ingredient(s) that make up the carrier, as well as any product that results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the disclosure and a pharmaceutically acceptable excipient, carrier or diluent.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration).

A “pharmaceutically acceptable salt” is a salt that can be formulated into a compound for pharmaceutical use, including but not limited to metal salts (e.g., sodium, potassium, magnesium, calcium, etc.) and salts of ammonia or organic amines. Examples of cysteamine salts include hydrochloride, bitartrate and phosphocysteamine derivatives. Cystamine and cystamine salts derivatives include sulfated cystamine.

As used herein “pharmaceutically acceptable” or “pharmacologically acceptable” salt, ester or other derivative of an active agent comprise, for example, salts, esters or other derivatives refers to a material that is not biologically or otherwise undesirable, i.e., the material may be administered to an individual without causing any undesirable biological effects or without interacting in a deleterious manner with any of the components of the composition in which it is contained or with any components present on or in the body of the individual.

As used herein, the term “unit dosage form” refers to physically discrete units suitable as unitary dosages for human and animal subjects, each unit containing a predetermined quantity of a compound of the disclosure calculated in an amount sufficient to produce the desired effect, optionally in association with a pharmaceutically acceptable excipient, diluent, carrier or vehicle. The specifications for the novel unit dosage forms of the present disclosure depend on the particular compound employed and the effect to be achieved, and the pharmacodynamics associated with each compound in the host.

As used herein, the term “subject” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender. In various embodiments the subject is human.

### Neurodegenerative Disease/Huntington's Disease

Huntington's Disease is often defined or characterized by onset of symptoms and progression of decline in motor and neurological function. HD can be broken into five stages: Patients with early HD (stages 1 and 2) have increasing concerns about cognitive issues, and these concerns remain constant during moderate/intermediate HD (stages 3 and 4). Patients with late-stage or advanced HD (stage 5) have a lack of cognitive ability (Ho et al., Clin Genet. September 2011; 80(3):235-239).

Progression of the stages can be observed as follows: Early Stage (stage 1), in which the person is diagnosed as having HD and can function fully both at home and work. Early Intermediate Stage (stage 2), the person remains employable but at a lower capacity and are able to manage their daily affairs with some difficulties. Late Intermediate Stage (stage 3), the person can no longer work and/or manage household responsibilities and. need help or supervision to handle daily financial and other daily affairs. Early Advanced Stage patients (stage 4) are no longer independent in daily activities but is still able to live at home supported by their family or professional careers. In the Advanced Stage (stage 5), the person requires complete support in daily activities and professional nursing care is usually needed. Patients with HD usually die about 15 to 20 years after their symptoms first appear.

In intermediate stages, as the disease progresses, the initial motor symptoms will gradually develop into more obvious involuntary movements such as jerking and twitching of the head, neck, arms and legs. These movements may interfere with walking, speaking and swallowing. People at this stage of Huntington's often look as if they're drunk: they stagger when they walk and their speech is slurred. They have increasing difficulty working or managing a household, but can still deal with most activities of daily living. The advanced stages of HD typically involve fewer involuntary movements and more rigidity. Patients in these stages of HD can no longer manage the activities of daily living. Difficulties with swallowing, communication and weight loss are common in the advanced stage.

Chorea is the most common movement disorder seen in HD. Initially, mild chorea resembles fidgetiness. Severe chorea may appear as uncontrollable flailing of the extremities. As the disease progresses, chorea gradually moves towards and is replaced by dystonia and parkinsonian features, such as bradykinesia, rigidity, and postural instability. In advanced disease, patients develop an akinetic-rigid syndrome, with minimal or no chorea. Other late features are spasticity, clonus, and extensor plantar responses. Dysarthria and dysphagia are common. Abnormal eye movements may be seen early in the disease. Other movement disorders, such as tics and myoclonus, may be seen in patients with HD. Juvenile HD (Westphal variant), defined as having an age of onset of younger than 20 years, is characterized by parkinsonian features, dystonia, long-tract signs, dementia, epilepsy, and mild or even absent chorea.

Cognitive decline is also characteristic of HD, and the rate of progression can vary among individual patients. Dementia and the psychiatric features of HD are often the earliest of functional impairment. Dementia syndrome associated with HD includes early onset behavioral changes, such as irritability, untidiness, and loss of interest, followed by slowing of cognition, impairment of intellectual function, and memory disturbances. This pattern corresponds well to the syndrome of subcortical dementia, and it has been suggested to reflect dysfunction of frontal-subcortical neuronal circuitry.

Early stages of HD are characterized by deficits in short-term memory, followed by motor dysfunction and a variety of cognitive changes in the intermediate stages of dementia (Loy et al., PLoS Curr. 2013; 5: Cleret de Langavant et al., PLoS One. 2013; 8(4):e61676). These deficits include diminished verbal fluency, problems with attention, executive function, visuospatial processing, and abstract reasoning. Language skills become affected in the final stages of the illness, resulting in marked word-retrieval deficiency.

HD can also manifest in behavioral disorders, including depression, with a small percentage of patients experiencing bouts of mania characteristic of bipolar disorder, an increased rate of suicide, and psychosis, obsessive-compulsive symptoms, sexual and sleep disorders, and changes in personality.

It is contemplated herein that administration of a cysteamine product or composition as described herein can alleviate and treat one or more symptoms associated with neurodegenerative disease. Such symptoms, include but are not limited to, one or motor skills, cognitive function, dystonia, chorea, psychiatric symptoms such as depression, brain and striatal atrophies, neuronal dysfunction,

It is contemplated that the administration results in a slower progression of total motor score compared to a subject not receiving cysteamine or a pharmaceutically acceptable salt thereof or cystamine or a pharmaceutically acceptable salt thereof. In some embodiments, the slower progression is a result in improvement in one or more motor scores selected from the group consisting of chorea subscore, balance and gait subscore, hand movements subscore, eye movement subscore and maximal dystonia subscore.

Additional indicia of a slower decline in symptoms of HD are measured using change from baseline in one or more of the following parameters: using standardized tests for (i) functional assessment (UHDRS Total Functional Capacity, Independence Scale); (ii) neuropsychological assessment (UHDRS Cognitive Assessment, Mattis Dementia Rating Scale, Trail Making Test A and B, Figure Cancellation Test, Hopkins Verbal Learning Test, Articulation Speed Test); and (iii) psychiatric assessment (UHDRS Behavioral Assessment, Montgomery and Asberg Depression Rating Scale).

In certain embodiments, alteration in one or more symptoms in patients receiving cysteamine or a pharmaceutically acceptable salt thereof or cystamine or a pharmaceutically acceptable salt thereof is shown to be beneficial by at least 10%, 20%, 25%, 30%, 35%, 40%, 45%, 50%, 55%, 60%, 65%, 70%, 75% or more compared to baseline assessment of the symptom. In certain embodiments, the rate of progression or decline in total motor score is slowed, by at least 20%, 25%, 30%, 35%, 40%, 45%, 50%, 55%, 60%, 65%, 70%, 75% or more. Measurement may be performed using the Unified Huntington Disease Rating Scale (UHDRS).

In certain embodiments, the symptoms are measured at 6 months, 12 months, 18 months or 2 years or more after administration.

The disclosure also provides a method for slowing the progression of brain and striatal atrophies and/or treating dystonia in a subject suffering from a neurodegenerative disease comprising administering to a subject in need thereof a composition comprising cysteamine or a pharmaceutically acceptable salt thereof or cystamine or a pharmaceutically acceptable salt thereof.

Also contemplated is a method for decreasing levels of transglutaminase in a subject suffering from a neurodegenerative disease comprising administering to a subject in need thereof a composition comprising cysteamine or a pharmaceutically acceptable salt thereof or cystamine or a pharmaceutically acceptable salt thereof. In various embodiments, the transglutaminase is Tgase 2.

It is contemplated that the neurodegenerative disease is Huntington's disease, including stage 1, stage 2, stage 3, stage 4 or stage 5 Huntington's Disease.

### Cysteamine/Cystamine

Cysteamine (HS—CH₂—CH₂—NH₂) is a small sulfhydryl compound that is able to cross cell membranes easily due to its small size. Cysteamine plays a role in formation of the protein glutathione (GSH) precursor, and is currently FDA approved for use in the treatment of cystinosis, an intra-lysosomal cystine storage disorder. In cystinosis, cysteamine acts by converting cystine to cysteine and cysteine-cysteamine mixed disulfide, which are then both able to leave the lysosome through the cysteine and lysine transporters respectively (Gahl et al., N Engl J Med 2002; 347(2):111-21). Within the cytosol the mixed disulfide can be reduced by its reaction with glutathione and the cysteine released can be used for further GSH synthesis. Treatment with cysteamine has been shown to result in lowering of intracellular cystine levels in circulating leukocytes (Dohil et al., J. Pediatr 148(6):764-9, 2006).

Cysteamine is also discussed in (Prescott et al., Lancet 1972; 2(7778):652; Prescott et al., Br Med J 1978; 1(6116):856-7; Mitchell et al., Clin Pharmacol Ther 1974; 16(4):676-84; Toxicol Appl Pharmacol. 1979 48(2):221-8; Qiu et al., World J Gastroenterol. 13:4328-32, 2007. Unfortunately, the sustained concentrations of cysteamine necessary for therapeutic effect are difficult to maintain due to rapid metabolism and clearance of cysteamine from the body, with nearly all administered cysteamine converted to taurine in a matter of hours. These difficulties are transferred to patients in the form of high dosing levels and frequencies, with all of the consequent unpleasant side effects associated with cysteamine (e.g., gastrointestinal distress and body odor) See the package insert for CYSTAGON® (cysteamine bitartrate). International Publication No. WO 2007/079670 discloses enterically coated cysteamine products and a method of reducing dosing frequency of cysteamine.

Cysteamine is addressed in International Patent Application Nos. WO 2009/070781, and WO 2007/089670, and U.S. Patent Publication Nos. 20110070272, 20090048154, and 20050245433.

Cysteamine plays a role in formation of the protein glutathione (GSH) precursor. In cystinosis, cysteamine acts by converting cystine to cysteine and cysteine-cysteamine mixed disulfide which are then both able to leave the lysosome through the cysteine and lysine transporters respectively (Gahl et al., N Engl J Med 2002; 347(2):111-21). Within the cytosol the mixed disulfide can be reduced by its reaction with glutathione and the cysteine released can be used for further GSH synthesis. The synthesis of GSH from cysteine is catalyzed by two enzymes, gamma-glutamylcysteine synthetase and GSH synthetase. This pathway occurs in almost all cell types, with the liver being the major producer and exporter of GSH. The reduced cysteine-cysteamine mixed disulfide will also release cysteamine, which, in theory is then able to re-enter the lysosome, bind more cystine and repeat the process (Dohil et al., J Pediatr 2006; 148(6):764-9). In a recent study in children with cystinosis, enteral administration of cysteamine resulted in increased plasma cysteamine levels, which subsequently caused prolonged efficacy in the lowering of leukocyte cystine levels (Dohil et al., J Pediatr 2006; 148(6):764-9). This may have been due to “re-cycling” of cysteamine when adequate amounts of drug reached the lysosome. If cysteamine acts in this fashion, then GSH production may also be significantly enhanced.

Cysteamine is a potent gastric acid-secretagogue that has been used in laboratory animals to induce duodenal ulceration; studies in humans and animals have shown that cysteamine-induced gastric acid hypersecretion is most likely mediated through hypergastrinemia. Cysteamine is currently FDA approved for use in the treatment of cystinosis, an intra-lysosomal cystine storage disorder. In previous studies performed in children with cystinosis who suffered regular upper gastrointestinal symptoms, a single oral dose of cysteamine (11-23 mg/kg) was shown to cause hypergastrinemia and a 2 to 3-fold rise in gastric acid-hypersecretion, and a 50% rise in serum gastrin levels. Symptoms suffered by these individuals included abdominal pain, heartburn, nausea, vomiting, and anorexia. U.S. patent application Ser. No. 11/990,869 and published International Publication No. WO 2007/089670 (each of which is incorporated by reference herein in its entirety) showed that cysteamine induced hypergastrinemia arises, in part, as a local effect on the gastric antral-predominant G-cells in susceptible individuals. The data also suggest that this is also a systemic effect of gastrin release by cysteamine. Depending on the route of administration, plasma gastrin levels usually peak after intragastric delivery within 30 minutes whereas the plasma cysteamine levels peak later.

Subjects with cystinosis ingest therapeutic oral cysteamine (CYSTAGON®) every 6 hours day and night, or use an enteric form of cysteamine (PROCYSBI®) every 12 hours. When taken regularly, cysteamine can deplete intracellular cystine by up to 90% (as measured in circulating white blood cells), and this had been shown to reduce the rate of progression to kidney failure/transplantation and also to obviate the need for thyroid replacement therapy. Because of the difficulty in taking CYSTAGON®, reducing the required dosing improves the adherence to therapeutic regimen. International Publication No. WO 2007/089670 demonstrates that delivery of cysteamine to the small intestine reduces gastric distress and ulceration and increases AUC. Delivery of cysteamine into the small intestine is useful due to improved absorption rates from the small intestine, and/or less cysteamine undergoing hepatic first pass elimination when absorbed through the small intestine. A decrease in leukocyte cystine was observed within an hour of treatment.

In addition, sulfhydryl (SH) compounds such as cysteamine, cystamine, and glutathione are active intracellular antioxidants. Cysteamine protects animals against bone marrow and gastrointestinal radiation syndromes. The rationale for the important anti-oxidant properties of SH compounds is further supported by observations in mitotic cells. These are the most sensitive to radiation injury in terms of cell reproductive death and are noted to have the lowest level of SH compounds. Conversely, S-phase cells, which are the most resistant to radiation injury using the same criteria, have demonstrated the highest levels of inherent SH compounds. In addition, when mitotic cells were treated with cysteamine, they became very resistant to radiation. It has also been noted that cysteamine may directly protect cells against induced mutations. The protection is thought to result from scavenging of free radicals, either directly or via release of protein-bound GSH. An enzyme that liberates cysteamine from coenzyme A has been reported in avian liver and hog kidney. Recently, studies have reported a protective effect of cysteamine against the hepatotoxic agents acetaminophen, bromobenzene, and phalloidine.

Cystamine, in addition to its role as a radioprotectant, has been found to alleviate tremors and prolong life in mice with the gene mutation for Huntington's disease (HD). The drug may work by increasing the activity of proteins that protect nerve cells, or neurons, from degeneration. However, due to the current methods and formulation of delivery of cystamine, degradation and poor uptake require excessive dosing.

### Cysteamine Products

In another aspect, the disclosure provides cysteamine products for use in the methods described herein.

A “cysteamine product” in the present disclosure refers generally to cysteamine or a pharmaceutically acceptable salt thereof or cystamine or a pharmaceutically acceptable salt thereof, including a biologically active metabolite or derivative thereof, or combination of cysteamine and cystamine, and includes cysteamine or cystamine salts, esters, amides, alkylate compounds, prodrugs, analogs, phosphorylated compounds, sulfated compounds, nitrosylated and glycosylated compounds or other chemically modified forms thereof (e.g., chemically modified forms prepared by labeling with radionucleotides or enzymes and chemically modified forms prepared by attachment of polymers such as polyethylene glycol). Thus, the cysteamine or cystamine can be administered in the form of a pharmacologically acceptable salt, ester, amide, prodrug or analog or as a combination thereof. In various embodiments, the cysteamine product includes cysteamine, cystamine or derivatives thereof. In any of the embodiments described herein, a cysteamine product may optionally exclude N-acetylcysteine.

Salts, esters, amides, prodrugs and analogs of the active agents may be prepared using standard procedures known to those skilled in the art of synthetic organic chemistry and described, for example, by J. March, “Advanced Organic Chemistry: Reactions, Mechanisms and Structure,” 4th Ed. (New York: Wiley-Interscience, 1992). For example, basic addition salts are prepared from the neutral drug using conventional means, involving reaction of one or more of the active agent's free hydroxyl groups with a suitable base. Generally, the neutral form of the drug is dissolved in a polar organic solvent such as methanol or ethanol and the base is added thereto. The resulting salt either precipitates or may be brought out of solution by addition of a less polar solvent. Suitable bases for forming basic addition salts include, but are not limited to, inorganic bases such as sodium hydroxide, potassium hydroxide, ammonium hydroxide, calcium hydroxide, trimethylamine, or the like. Preparation of esters involves functionalization of hydroxyl groups which may be present within the molecular structure of the drug. The esters are typically acyl-substituted derivatives of free alcohol groups, i.e., moieties which are derived from carboxylic acids of the formula R—COOH where R is alkyl, and typically is lower alkyl. Esters can be reconverted to the free acids, if desired, by using conventional hydrogenolysis or hydrolysis procedures. Preparation of amides and prodrugs can be carried out in an analogous manner. Other derivatives and analogs of the active agents may be prepared using standard techniques known to those skilled in the art of synthetic organic chemistry, or may be deduced by reference to the pertinent literature.

In various embodiments, the cysteamine product does not refer to nanoparticles (including, but not limited to, gold, silver, cadmium and iron nanoparticles) comprising cysteamine (e.g., Wu et al., Nanomedicine: Nanotechnology, Biology and Medicine, 8:860,869, 2011; Ghosh et al., Biomaterials, 34:807-816, 2013; Unak et al., Surf. N. Niointerfaces, 90:217-226, 2012; Petkova et al, m Nanoscale Res. Lett., 7:287, 2012; and U.S. Patent Publication No. 2010/0034735 or cysteamine incorporated into another active agent (e.g., Fridkin et al., J. Comb. Chem., 7:977-986, 2005).

### Pharmaceutical Formulations

The disclosure provides cysteamine products useful in the treatment of neurodegenerative disease, such as Huntington's Disease (e.g., to slow or improve motor skills, cognitive function and promote neuronal regeneration). To administer cysteamine products to patients or test animals, it is preferable to formulate the cysteamine products in a composition comprising one or more pharmaceutically acceptable carriers. Pharmaceutically or pharmacologically acceptable carriers or vehicles refer to molecular entities and compositions that do not produce allergic, or other adverse reactions when administered using routes well-known in the art, as described below, or are approved by the U.S. Food and Drug Administration or a counterpart foreign regulatory authority as an acceptable additive to orally or parenterally administered pharmaceuticals. Pharmaceutically acceptable carriers include any and all clinically useful solvents, dispersion media, coatings, antibacterial and antifungal agents, isotonic and absorption delaying agents and the like.

Pharmaceutical carriers include pharmaceutically acceptable salts, particularly where a basic or acidic group is present in a compound. For example, when an acidic substituent, such as —COOH, is present, the ammonium, sodium, potassium, calcium and the like salts, are contemplated for administration. Additionally, where an acid group is present, pharmaceutically acceptable esters of the compound (e.g., methyl, tert-butyl, pivaloyloxymethyl, succinyl, and the like) are contemplated as preferred forms of the compounds, such esters being known in the art for modifying solubility and/or hydrolysis characteristics for use as sustained release or prodrug formulations.

When a basic group (such as amino or a basic heteroaryl radical, such as pyridyl) is present, then an acidic salt, such as hydrochloride, hydrobromide, acetate, maleate, pamoate, phosphate, methanesulfonate, p-toluenesulfonate, and the like, is contemplated as a form for administration.

In addition, compounds may form solvates with water or common organic solvents. Such solvates are contemplated as well.

The cysteamine products may be administered orally, parenterally, transocularly, intranasally, transdermally, transmucosally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well. Generally, compositions for administration by any of the above methods are essentially free of pyrogens, as well as other impurities that could be harmful to the recipient. Further, compositions for administration parenterally are sterile.

Pharmaceutical compositions of the disclosure containing a cysteamine product, e.g., cyteamine bitartrate, as an active ingredient may contain pharmaceutically acceptable carriers or additives depending on the route of administration. Examples of such carriers or additives include water, a pharmaceutically acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the disclosure.

Formulation of the pharmaceutical composition will vary according to the route of administration selected (e.g., solution, emulsion). An appropriate composition comprising the cysteamine product to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., water, buffered water, 0.4% saline, 0.3% glycine, or aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

In some embodiments, the cysteamine product disclosed herein can be lyophilized for storage and reconstituted in a suitable carrier prior to use. Any suitable lyophilization and reconstitution techniques can be employed. It is appreciated by those skilled in the art that lyophilization and reconstitution can lead to varying degrees of activity loss and that use levels may have to be adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

In one embodiment, the disclosure provides use of an enterically coated cysteamine product composition, e.g., cysteamine bitartrate. Enteric coatings prolong release until the cysteamine product reaches the intestinal tract, typically the small intestine. Because of the enteric coatings, delivery to the small intestine is improved thereby improving uptake of the active ingredient while reducing gastric side effects. Exemplary enterically coated cysteamine products are described in International Publication No. WO 2007/089670 and in International Patent Applications PCT/US14/42607 and PCT/US14/42616.

In some embodiments, the coating material is selected such that the therapeutically active agent is released when the dosage form reaches the small intestine or a region in which the pH is greater than pH 4.5. In various embodiments, the formulation releases at a pH of about 4.5 to 6.5, 4.5 to 5.5, 5.5 to 6.5 or about pH 4.5, 5.0, 5.5, 6.0 or 6.5.

The coating may be a pH-sensitive materials, which remain intact in the lower pH environs of the stomach, but which disintegrate or dissolve at the pH commonly found in the small intestine of the patient. For example, the enteric coating material begins to dissolve in an aqueous solution at pH between about 4.5 to about 5.5. For example, pH-sensitive materials will not undergo significant dissolution until the dosage form has emptied from the stomach. The pH of the small intestine gradually increases from about 4.5 to about 6.5 in the duodenal bulb to about 7.2 in the distal portions of the small intestine. In order to provide predictable dissolution corresponding to the small intestine transit time of about 3 hours (e.g., 2-3 hours) and permit reproducible release therein, the coating should begin to dissolve at the pH range within the small intestine. Therefore, the amount of enteric polymer coating should be sufficient to substantially dissolved during the approximate three hour transit time within the small intestine, such as the proximal and mid-intestine.

Enteric coatings have been used for many years to arrest the release of the drug from orally ingestible dosage forms. Depending upon the composition and/or thickness, the enteric coatings are resistant to stomach acid for required periods of time before they begin to disintegrate and permit release of the drug in the lower stomach or upper part of the small intestines. Examples of some enteric coatings are disclosed in U.S. Pat. No. 5,225,202 which is incorporated by reference fully herein. As set forth in U.S. Pat. No. 5,225,202, some examples of coating previously employed are beeswax and glyceryl monostearate; beeswax, shellac and cellulose; and cetyl alcohol, mastic and shellac, as well as shellac and stearic acid (U.S. Pat. No. 2,809,918); polyvinyl acetate and ethyl cellulose (U.S. Pat. No. 3,835,221); and neutral copolymer of polymethacrylic acid esters (Eudragit L30D) (F. W. Goodhart et al. , Pharm. Tech., pp. 64-71, April 1984); copolymers of methacrylic acid and methacrylic acid methylester (Eudragits), or a neutral copolymer of polymethacrylic acid esters containing metallic stearates (Mehta et al., U.S. Pat. Nos. 4,728,512 and 4,794,001). Such coatings comprise mixtures of fats and fatty acids, shellac and shellac derivatives and the cellulose acid phthlates, e.g., those having a free carboxyl content. See, Remington's at page 1590, and Zeitova et al. (U.S. Pat. No. 4,432,966), for descriptions of suitable enteric coating compositions. Accordingly, increased adsorption in the small intestine due to enteric coatings of cysteamine product compositions can result in improved efficacy.

Generally, the enteric coating comprises a polymeric material that prevents cysteamine product release in the low pH environment of the stomach but that ionizes at a slightly higher pH, typically a pH of 4 or 5, and thus dissolves sufficiently in the small intestines to gradually release the active agent therein. Accordingly, among the most effective enteric coating materials are polyacids having a pKa in the range of about 3 to 5. Suitable enteric coating materials include, but are not limited to, polymerized gelatin, shellac, methacrylic acid copolymer type CNF, cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethylcellulose (CMEC), hydroxypropyl methylcellulose acetate succinate (HPMCAS), and acrylic acid polymers and copolymers, typically formed from methyl acrylate, ethyl acrylate, methyl methacrylate and/or ethyl methacrylate with copolymers of acrylic and methacrylic acid esters (Eudragit NE, Eudragit RL, Eudragit RS). In one embodiment, the cysteamine product composition is administered in an oral delivery vehicle, including but not limited to, tablet or capsule form. Tablets are manufactured by first enterically coating the cysteamine product. A method for forming tablets herein is by direct compression of the powders containing the enterically coated cysteamine product, optionally in combination with diluents, binders, lubricants, disintegrants, colorants, stabilizers or the like. As an alternative to direct compression, compressed tablets can be prepared using wet-granulation or dry-granulation processes. Tablets may also be molded rather than compressed, starting with a moist material containing a suitable water-soluble lubricant.

The preparation of delayed, controlled or sustained/extended release forms of pharmaceutical compositions with the desired pharmacokinetic characteristics is known in the art and can be accomplished by a variety of methods. For example, oral controlled delivery systems include dissolution-controlled release (e.g., encapsulation dissolution control or matrix dissolution control), diffusion-controlled release (reservoir devices or matrix devices), ion exchange resins, osmotic controlled release or gastroretentive systems. Dissolution controlled release can be obtained, e.g., by slowing the dissolution rate of a drug in the gastrointestinal tract, incorporating the drug in an in soluble polymer, and coating drug particles or granules with polymeric materials of varying thickness. Diffusion controlled release can be obtained, e.g., by controlling diffusion through a polymeric membrane or a polymeric matrix. Osmotically controlled release can be obtained, e.g., by controlling solvent influx across a semipermeable membrane, which in turn carries the drug outside through a laser-drilled orifice. The osmotic and hydrostatic pressure differences on either side of the membrane govern fluid transport. Prolonged gastric retention may be achieved by, e.g., altering density of the formulations, bioadhesion to the stomach lining, or increasing floating time in the stomach. For further detail, see the Handbook of Pharmaceutical Controlled Release Technology, Wise, ed., Marcel Dekker, Inc., New York, N.Y. (2000), incorporated by reference herein in its entirety, e.g. Chapter 22 (“An Overview of Controlled Release Systems”).

The concentration of cysteamine product in these formulations can vary widely, for example from less than about 0.5%, usually at or at least about 1% to as much as 15 or 20% by weight and are selected primarily based on fluid volumes, manufacturing characteristics, viscosities, etc., in accordance with the particular mode of administration selected. Actual methods for preparing administrable compositions are known or apparent to those skilled in the art and are described in more detail in, for example, Remington's Pharmaceutical Science, 15th ed., Mack Publishing Company, Easton, Pa. (1980) and further editions thereof.

Compositions useful for administration may be formulated with uptake or absorption enhancers to increase their efficacy. Such enhancers include, for example, salicylate, glycocholate/linoleate, glycholate, aprotinin, bacitracin, SDS, caprate and the like. See, e.g., Fix (J. Pharm. Sci., 85:1282-1285, 1996) and Oliyai and Stella (Ann. Rev. Pharmacol. Toxicol., 32:521-544, 1993).

The enterically coated cysteamine product can comprise various excipients, as is well known in the pharmaceutical art, provided such excipients do not exhibit a destabilizing effect on any components in the composition. Thus, excipients such as binders, bulking agents, diluents, disintegrants, lubricants, fillers, carriers, and the like can be combined with the cysteamine product. Oral delivery vehicles contemplated for use herein include tablets, capsules, comprising the product. For solid compositions, diluents are typically necessary to increase the bulk of a tablet or capsule so that a practical size is provided for compression. Suitable diluents include dicalcium phosphate, calcium sulfate, lactose, cellulose, kaolin, mannitol, sodium chloride, dry starch and powdered sugar. Binders are used to impart cohesive qualities to a oral delivery vehicle formulation, and thus ensure that a tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, hydroxyethyl cellulose, hypromellose, and the like), and Veegum. Lubricants are used to facilitate oral delivery vehicle manufacture; examples of suitable lubricants include, for example, magnesium stearate, calcium stearate, and stearic acid, and are typically present at no more than approximately 1 weight percent relative to tablet weight. Disintegrants are used to facilitate oral delivery vehicle, (e.g., a tablet) disintegration or “breakup” after administration, and are generally starches, clays, celluloses, algins, gums or crosslinked polymers. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. If desired, flavoring, coloring and/or sweetening agents may be added as well. Other optional components for incorporation into an oral formulation herein include, but are not limited to, preservatives, suspending agents, thickening agents, and the like. Fillers include, for example, insoluble materials such as silicon dioxide, titanium oxide, alumina, talc, kaolin, powdered cellulose, microcrystalline cellulose, and the like, as well as soluble materials such as mannitol, urea, sucrose, lactose, dextrose, sodium chloride, sorbitol, and the like.

A pharmaceutical composition may also comprise a stabilizing agent such as hydroxypropyl methylcellulose or polyvinylpyrrolidone, as disclosed in U.S. Pat. No. 4,301,146. Other stabilizing agents include, but are not limited to, cellulosic polymers such as hydroxypropyl cellulose, hydroxyethyl cellulose, methyl cellulose, ethyl cellulose, cellulose acetate, cellulose acetate phthalate, cellulose acetate trimellitate, hydroxypropyl methylcellulose phthalate, microcrystalline cellulose and carboxymethylcellulose sodium; and vinyl polymers and copolymers such as polyvinyl acetate, polyvinylacetate phthalate, vinylacetate crotonic acid copolymer, and ethylene-vinyl acetate copolymers. The stabilizing agent is present in an amount effective to provide the desired stabilizing effect; generally, this means that the ratio of cysteamine product to the stabilizing agent is at least about 1:500 w/w, more commonly about 1:99 w/w.

The tablet, capsule, or other oral delivery system is manufactured by enterically coating the cysteamine product. A method for forming tablets herein is by direct compression of the powders containing the enterically coated cysteamine product, optionally in combination with diluents, binders, lubricants, disintegrants, colorants, stabilizers or the like. As an alternative to direct compression, compressed tablets can be prepared using wet-granulation or dry-granulation processes. Tablets may also be molded rather than compressed, starting with a moist material containing a suitable water-soluble lubricant.

In various embodiments, the enterically coated cysteamine product is granulated and the granulation is compressed into a tablet or filled into a capsule. In certain embodiments, the granules are enterically coated prior to compressing into a tablet or capsule. Capsule materials may be either hard or soft, and are typically sealed, such as with gelatin bands or the like. Tablets and capsules for oral use will generally include one or more commonly used excipients as discussed herein.

In a further embodiment, the cystemine product is formulated as a capsule. In one embodiment, the capsule comprises the cysteamine product and the capsule is then enterically coated. Capsule formulations are prepared using techniques known in the art.

A suitable pH-sensitive polymer is one which will dissolve in intestinal environment at a higher pH level (pH greater than 4.5), such as within the small intestine and therefore permit release of the pharmacologically active substance in the regions of the small intestine and not in the upper portion of the GI tract, such as the stomach.

In various embodiments, exemplary cysteamine or cystamine product formulations contemplated for use in the present methods are described in International Patent Applications PCT/US 14/42607 and PCT/US 14/42616.

For administration of the dosage form, i.e., the tablet or capsule comprising the enterically coated cysteamine product, a total weight in the range of approximately 100 mg to 1000 mg is used. In various embodiments, the tablet or capsule comprises 25, 50, 75, 100, 125, 150, 175, 200, 225, 250, 275, 300, 400 or 500 mg active ingredient, and multiple tablets or capsules are administered to reach the desired dosage. The dosage form is orally administered to a subject need thereof.

In addition, various prodrugs can be “activated” by use of the enterically coated cysteamine. Prodrugs are pharmacologically inert, they themselves do not work in the body, but once they have been absorbed, the prodrug decomposes. The prodrug approach has been used successfully in a number of therapeutic areas including antibiotics, antihistamines and ulcer treatments. The advantage of using prodrugs is that the active agent is chemically camouflaged and no active agent is released until the drug has passed out of the gut and into the cells of the body. For example, a number of produgs use S—S bonds. Weak reducing agents, such as cysteamine, reduce these bonds and release the drug. Accordingly, the compositions of the disclosure are useful in combination with pro-drugs for timed release of the drug. In this aspect, a pro-drug can be administered followed by administration of an enterically coated cysteamine compositions of the disclosure (at a desired time) to activate the pro-drug.

Prodrugs of cysteamine have been described previously. See, e.g., Andersen et al., In Vitro Evaluation of Novel Cysteamine Prodrugs Targeted to g-Glutamyl Transpeptidase (poster presentation), which describes S-pivaloyl cysteamine derivatives, S-benzoyl cysteamine derivatives, S-acetyl cysteamine derivatives and S-benzoyl cysteamine)glutamate-ethyl ester). Omran et al., Bioorg Med Chem Lett. 2011 Apr. 15; 21(8):2502-4 describes a folate pro-drug of cystamine as a treatment for nephropathic cystinosis.

Thiazolidine prodrugs are also contemplated, and can be made as described previously. See e.g., Wilmore et al., J. Med. Chem., 44 (16):2661-2666, 2001 and Cardwell, W A, “Synthesis And Evaluation Of Novel Cysteamine Prodrugs” 2006, Thesis, Univ. of Sunderland.

### Dosing and Administration

The cysteamine product is administered in a therapeutically effective amount; typically, the composition is in unit dosage form. The amount of cysteamine product administered is, of course, dependent on the age, weight, and general condition of the patient, the severity of the condition being treated, and the judgment of the prescribing-physician. Suitable therapeutic amounts will be known to those skilled in the art and/or are described in the pertinent reference texts and literature. Current non-enterically coated doses are about 1.35 g/m² body surface area and are administered 4-5 times per day (Levtchenko et al., Pediatr Nephrol. 21:110-113, 2006). In one aspect, the dose is administered either one time per day or multiple times per day.

The cysteamine product may be administered less than four time per day, e.g., one, two or three times per day. In various embodiments, the total daily dose of cysteamine or a pharmaceutically acceptable salt thereof or cystamine or a pharmaceutically acceptable salt thereof for treatment of huntongton's Disease or other indication described herein is between 500 to 2000 mg, 750 to 1750 mg, 1000 to 1500 mg, or may range between any two of the foregoing values. In various embodiments, the total daily dose of cysteamine or a pharmaceutically acceptable salt thereof or cystamine or a pharmaceutically acceptable salt thereof is 500, 600, 700, 800, 900, 1000, 1100, 1200, 1300, 1400, 1500, 1600, 1700, 1800, 1900 or 2000 mg per day. It is contemplated that any of the foregoing doses is administered twice daily. It is further contemplated that any of the foregoing doses is administered in two equal doses daily. Optionally, the daily dose is administered in three doses.

In some embodiments, an effective dosage of cysteamine product may be within the range of 0.01 mg to 1000 mg per kg (mg/kg) of body weight per day. In some embodiments, the cysteamine, cystamine or pharmaceutically acceptable salt thereof is administered at a daily dose ranging from about 10 mg/kg to about 250 mg/kg, or from about 100 mg/kg to about 250 mg/kg, or from about 60 mg/kg to about 100 mg/kg or from about 50 mg/kg to about 90 mg/kg, or from about 30 mg/kg to about 80 mg/kg, or from about 20 mg/kg to about 60 mg/kg, or from about 10 mg/kg to about 50 mg/kg, or from about 15 to about 25 mg/kg, or from about 15 to about 20 mg/kg or from about 10 to about 20 mg/kg. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 225 mg/kg, 250 mg/kg, 275 mg/kg, 300 mg/kg, 325 mg/kg, 350 mg/kg, 375 mg/kg, 400 mg/kg, 425 mg/kg, 450 mg/kg, 475 mg/kg, 500 mg/kg, 525 mg/kg , 550 mg/kg, 575 mg/kg, 600 mg/kg, 625 mg/kg, 650 mg/kg, 675 mg/kg, 700 mg/kg, 725 mg/kg, 750 mg/kg, 775 mg/kg, 800 mg/kg, 825 mg/kg, 850 mg/kg, 875 mg/kg, 900 mg/kg, 925 mg/kg, 950 mg/kg, 975 mg/kg or 1000 mg/kg, or may range between any two of the foregoing values.

In some embodiments, the cysteamine product is administered at a total daily dose of from approximately 0.25 g/m² to 4.0 g/m² body surface area, e.g., at least about 0.3, 0.4, 0.5, 0.6, 0.7, 0.8, 0.9, 1.0, 1.1, 1.2, 1.3, 1.4, 1.5, 1.6, 1.7, 1.8, 1.9 or 2 g/m², or up to about 0.8, 0.9, 1.0, 1.1, 1.2, 1.3, 1.4, 1.5, 1.6, 1.7, 1.8, 1.9, 2.0, 2.2, 2.5, 2.7, 3.0, 3.25, 3.5 or 3.75 g/m² or may range between any two of the foregoing values. In some embodiments, the cysteamine product may be administered at a total daily dose of about 0.5-2.0 g/m² body surface area, or 1-1.5 g/m² body surface area, or 1-1.95g/m² body surface area, or 0.5-1 g/m² body surface area, or about 0.7-0.8 g/m² body surface area, or about 1.35 g/m² body surface area, or about 1.3 to about 1.95 grams/m2/day, or about 0.5 to about 1.5 grams/m2/day, or about 0.5 to about 1.0 grams/m2/day, preferably at a frequency of fewer than four times per day, e.g. three, two or one times per day. Salts or esters of the same active ingredient may vary in molecular weight depending on the type and weight of the salt or ester moiety. For administration of enteric dosage form, e.g., a tablet or capsule or other oral dosage form comprising the enterically coated cysteamine product, a total weight in the range of approximately 100 mg to 1000 mg is used. In various embodiments, the tablet or capsule comprises 25, 50, 75, 100, 125, 150, 175, 200, 225, 250, 275, 300, 400 or 500 mg active ingredient, and multiple tablets or capsules are administered to reach the desired dosage

Administration may continue for at least 3 months, 6 months, 9 months, 1 year, 2 years, or more.

### Combination Therapy

Therapeutic compositions can be administered in therapeutically effective dosages alone or in combination with adjunct therapy such as antipsychotics, antidepressants, vesicular monoamine transporter (VMAT)-inhibitors such as tetrabenazine, dopamine inhibitors, laquinimod, CNS-immunomodulators, neuroprotective factors, BDNF and agents that upregulate BDNF, ampakines, positive modulators of AMPA-type glutamate receptors, activators of BDNF receptor TrkB and gene therapy.

Antidepressants include: SSRI antidepressants, such as fluoxetine, citalopram and paroxetine, tricyclic antidepressants, such as amitriptyline, other types of antidepressants, including mirtazapine, duloxetine and venlafaxine.

Antipsychotic medication includes risperidone, olanzapine, aripiprazole, tiapride and quetiapine, benzodiazepines, such as clonazepam and diazepam, and mood stabilizers, such as carbamazepine.

In some embodiments, the methods (or uses) described herein further comprise administering a further therapeutic agent selected from the group consisting of tetrabenazine, laquinimod, BDNF, ampakines, fluoxetine, citalopram, paroxetine, amitriptyline, mirtazapine, duloxetine, venlafaxine, risperidone, olanzapine, aripiprazole, tiapride, quetiapine, clonazepam diazepam and carbamazepine.

The cysteamine product and other drugs/therapies can be administered in combination either simultaneously in a single composition or in separate compositions. Alternatively, the administration is sequential. Simultaneous administration is achieved by administering a single composition or pharmacological protein formulation that includes both the cysteamine product and other therapeutic agent(s). Alternatively, the other therapeutic agent(s) are taken separately at about the same time as a pharmacological formulation (e.g., tablet, injection or drink) of the cysteamine product.

In various alternatives, administration of the cysteamine product can precede or follow administration of the other therapeutic agent(s) by intervals ranging from minutes to hours. For example, in various embodiments, it is further contemplated that the agents are administered in a separate formulation and administered concurrently, with concurrently referring to agents given within 30 minutes of each other.

In embodiments where the other therapeutic agent(s) and the cysteamine product are administered separately, one would generally ensure that the cysteamine product and the other therapeutic agent(s) are administered within an appropriate time of one another so that both the cysteamine product and the other therapeutic agent(s) can exert, synergistically or additively, a beneficial effect on the patient. For example, in various embodiments the cysteamine product is administered within about 0.5-6 hours (before or after) of the other therapeutic agent(s). In various embodiments, the cysteamine product is administered within about 1 hour (before or after) of the other therapeutic agent(s).

In another aspect, the second agent is administered prior to administration of the cysteamine composition. Prior administration refers to administration of the second agent within the range of one week prior to treatment with cysteamine, up to 30 minutes before administration of cysteamine. It is further contemplated that the second agent is administered subsequent to administration of the cysteamine composition. Subsequent administration is meant to describe administration from 30 minutes after cysteamine treatment up to one week after cysteamine administration.

In various embodiments, the effects of cysteamine products on the symptoms of Huntington's Disease or other neurological disease as described herein are measured as improvements in disease symptoms described above, or are measured as a slowing or decrease in the time of progression of a disease symptom, e.g., a slowed progression of total motor score can be considered an improvement in a disease symptom.

### Kits

The disclosure also provides kits for carrying out the methods of the disclosure. In various embodiments, the kit contains, e.g., bottles, vials, ampoules, tubes, cartridges and/or syringes that comprise a liquid (e.g., sterile injectable) formulation or a solid (e.g., lyophilized) formulation. The kits can also contain pharmaceutically acceptable vehicles or carriers (e.g., solvents, solutions and/or buffers) for reconstituting a solid (e.g., lyophilized) formulation into a solution or suspension for administration (e.g., by injection), including without limitation reconstituting a lyophilized formulation in a syringe for injection or for diluting concentrate to a lower concentration. Furthermore, extemporaneous injection solutions and suspensions can be prepared from, e.g., sterile powder, granules, or tablets comprising a cysteamine product-containing composition. The kits can also include dispensing devices, such as aerosol or injection dispensing devices, pen injectors, autoinjectors, needleless injectors, syringes, and/or needles. In various embodiments, the kit also provides an oral dosage form, e.g., a tablet or capsule or other oral formulation described herein, of the cysteamine product for use in the method. The kit also provides instructions for use.

While the disclosure has been described in conjunction with specific embodiments thereof, the foregoing description as well as the examples which follow are intended to illustrate and not limit the scope of the disclosure. Other aspects, advantages and modifications within the scope of the disclosure will be apparent to those skilled in the art.

### EXAMPLES

### Example 1

### Protocol for Clinical Trial

Study Organization

In this double-blind, placebo-controlled multi-center trial, the efficacy, safety, and tolerability of entereically coated cysteamine in modifying HD progression was assessed, as measured by change in Total Motor Score (TMS) of the Unified Huntington's Disease Rating Scale (UHCRS). Patients were recruited from nine departments of neurology and genetics throughout France. The protocol and consent forms were approved by an institutional review board in accordance with French law.

Participants

Male and female HD patients, aged between 18 and 65 years, in whom the number of CAG repeats in the HTT gene was >38 were enrolled. Inclusion criteria were a minimum score for two components of the Unified Huntington Disease Rating Scale (UHDRS)(18): Total Motor Score (TMS)≧5 and Total Functional Capacity (TFC)>10. The UHDRS is a validated scale that assesses four domains of clinical performance and capacity: motor function, cognitive function, behavioral abnormalities, and functional capacity (18). The TMS is the sum of 31 items in multiple domains of motor impairment: oculomotor function, dysarthria, chorea, dystonia, and gait and postural stability. Additional criteria for measuring motor score include hand movements, bradykinesia-body or arm rigidity, and tongue protrusion. The sum of motor scores can range from 0 to 124 and higher scores indicate more impaired motor function. The TFC rates the person's level of independence and scores range from 0 to 13 with greater scores indicating higher functioning.

All patients were allowed to continue their baseline medication regimen throughout the study, including antidepressants, tetrabenazine and other antipsychotics such as olanzapine, aripiprazole, risperidone, and tiapride. Written, informed consent was obtained from all patients before the start of any study-related procedures.

Study Procedures

Based on a previous publication (19) and a previous 4-month trial in patients affected with HD (unpublished data), it was decided to use a delayed-release formulation of cysteamine bitartrate (RP103) at 70% of the maximum tolerated dose of the immediate-release formulation (20,21). Eligible patients were randomized in a double-blind, 1:1 ratio to receive placebo or 600 mg of RP103 orally every 12 hours for 18 months. Patients were then enrolled in the still ongoing 18-month open phase of this study.

The primary endpoint of the study was change in the TMS of the UHDRS from baseline to 18 months. The TMS was assessed in each patient at the run-in visit (M-1), at baseline and at 12 and 18 months for each patient.

Secondary endpoints of the study were change from baseline to 18 months using standardized tests for (i) functional assessment (UHDRS Total Functional Capacity, Independence Scale); (ii) neuropsychological assessment (UHDRS Cognitive Assessment, Mattis Dementia Rating Scale, Trail Making Test A and B, Figure Cancellation Test, Hopkins Verbal Learning Test, Articulation Speed Test, categorical verbal fluency, figure cancellation test); and (iii) psychiatric assessment (UHDRS Behavioral Assessment, Montgomery and Asberg Depression Rating Scale, MADRS; and brain-derived neurotrophic factor (BDNF) concentration).

Safety and tolerability were evaluated throughout the study by clinical assessment, laboratory tests and reports of adverse events.

Blood samples for evaluation of cysteamine and brain-derived neurotrophic factor (BDNF) concentration were performed just before the dose for RP103 or placebo at baseline and at 6, 12 and 18 months.

Intent-to-Treat and Per-Protocol Population

The intent-to-treat (ITT) population included all randomised patients and corresponds to the primary population for the analysis of all efficacy endpoints. The per-protocol (PP) population comprises all patients from the ITT population who did not have any major protocol violations. The safety population is defined as all patients who received at least one dose of study medication and is used for the analysis of safety endpoints.

All 96 enrolled patients were included in the intent-to-treat (ITT). As defined in the statistical plan, the per-protocol set of patients included the 85 patients without major protocol deviations, 43 patients in the placebo group and 42 in the RP103 group.

Due to the 18-month duration of the study, patients were allowed to continue their baseline medication regimen, including antidepressants, tetrabenazine (the only approved medication to treat chorea associated with HD), and antipsychotics such as olanzapine, aripiprazole, risperidone, and tiapride. Because tetrabenazine is administered to treat chorea, which is one of the subscore tests of the TMS, to control for possible effects of tetrabenazine on TMS results, a post-hoc subgroup analysis from per-protocol population was also performed on those patients not treated with tetrabenazine at any time during the study (NoTBZ group) for primary and secondary endpoints.

Statistical Analysis

The estimation of sample size was based on the data collected by the HD French-speaking Network in which the mean annual TMS progression was +13.0 (±14.0)22. Assuming a potential 30-40% dropout rate, it was estimated that 96 patients would have to undergo randomization for the study to reach 95% power to show a significant difference between groups, assuming an average difference of −13.0 in the change between RP103 group and the placebo group with a standard deviation of 14.022.

Statistical analyses were performed using a statistical analysis plan approved before locking the database. Efficacy analyses were done with a general repeated mixed model that included the following covariates: center, number of CAG triplet repeats, age and body mass index (BMI) for UHDRS motor scores. Center was a factor of stratification in the randomization, and age, CAG count, and BMI were included due to their expected moderate to important influence on motor, functional, or psychological questionnaires. For functional and psychological endpoints, sex was added as a covariate due to its potential influence in response. Nonparametric analysis of covariance stratified by center (van Elteren test) was performed for non-normally distributed endpoints. Treatment effects were estimated using the Hodges-Lehmann method. All statistical tests were two-sided, with a 5% level of significance. All models adjusted for the baseline value. In order to display primary and secondary endpoints visually on forest plots, general mixed linear models were performed on standardized values; that is, each individual's values were adjusted with their mean baseline and divided by the standard deviation at baseline of the total group for each endpoint. This approach is valid under the assumption of normality of endpoints and was conducted on parameters for which a normal distribution could be assumed.

### Example 2

### Twice Daily Cysteamine Improves Motor Score in HD Patients

From October 2010 to June 2012, a total of 96 subjects were randomized to treatment and constituted the ITT cohort. The baseline characteristics of the ITT and NoTBZ populations are shown in Table 1.

TABLE 1

| | ITT | ITT | Overall | NoTBZ | NoTBZ |
|---|---|---|---|---|---|
| | placebo | RP103 | ITT | Placebo | RP103 |
| Characteristic | (N = 45) | (N = 51) | (N = 96) | (N = 32) | (N = 34) |
| Age (years, mean ± sd) | 49.7 ± 9.7 | 45.8 ± 8.9 | 47.6 ± 9.4 | 51.3 ± 10.0 | 45.3 ± 8.2 |
| Female (n, %) | 25 (56%) | 21 (41%) | 46 (48%) | 16 (50%) | 14 (41%) |
| Weight (kg, mean ± sd) | 68 ± 16 | 68 ± 13 | 68 ± 15 | 68 ± 15 | 68 ± 12 |
| Age at onset (years, | 44.6 ± 9.4 | 41.5 ± 8.4 | 42.9 ± 9.0 | 46.4 ± 9.3 | 40.6 ± 8.2 |
| mean ± sd) | | | | | |
| Time since onset (years, | 5.0 ± 2.9 | 4.3 ± 3.6 | 4.6 ± 3.3 | 4.9 ± 3.1 | 4.7 ± 3.6 |
| mean ± sd) | | | | | |
| CAG repeats (mean ± sd) | 43.6 ± 2.8 | 44.6 ± 2.8 | 44.2 ± 2.8 | 43.2 ± 2.8 | 44.6 ± 2.7 |
| UHDRS TMS (mean ± sd) | 24.9 ± 10.4 | 23.5 ± 11.0 | 24.2 ± 10.7 | 24.0 ± 9.8 | 24.2 ± 12.1 |
| Total functional capacity | 11.7 ± 0.8 | 11.9 ± 0.8 | 11.8 ± 0.8 | 11.7 ± 0.7 | 11.9 ± 0.9 |
| (mean ± sd) | | | | | |
| MATTIS total score | 132.4 ± 8.5 | 133.5 ± 7.7 | 133.0 ± 8.0 | 132.6 ± 8.5 | 134.7 ± 6.7 |
| (mean ± sd) | | | | | |
| Verbal fluency 1 min | 25.0 ± 9.4 | 25.6 ± 11.6 | 25.3 ± 10.6 | 26.1 ± 10.4 | 28.1 ± 11.2 |
| (mean ± sd) | | | | | |
| Symbol digit modalities | 30.3 ± 10.9 | 31.4 ± 10.2 | 30.9 ± 10.5 | 30.8 ± 10.9 | 33.6 ± 10.4 |
| test (mean ± sd) | | | | | |
| Stroop color naming | 52.3 ± 14.6 | 53.9 ± 10.4 | 53.1 ± 12.5 | 50.3 ± 15.1 | 54.4 ± 10.8 |
| (mean ± sd) | | | | | |
| Stroop word reading | 68.0 ± 16.7 | 71.6 ± 13.0 | 69.9 ± 14.8 | 66.0 ± 16.5 | 74.4 ± 13.4 |
| (mean ± sd) | | | | | |
| Stroop interference | 28.9 ± 9.7 | 32.1 ± 8.4 | 30.6 ± 9.1 | 28.1 ± 9.7 | 32.4 ± 9.1 |
| (mean ± sd) | | | | | |

Efficacy on TMS

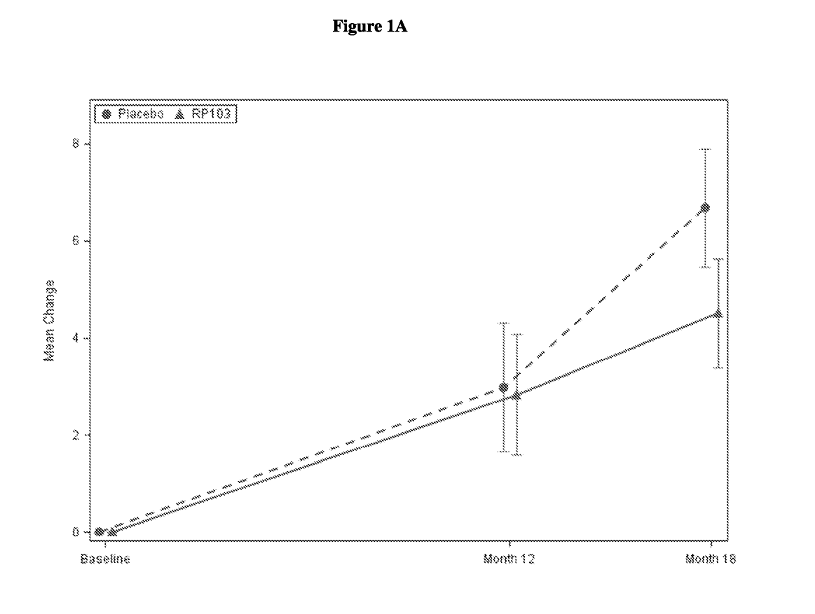

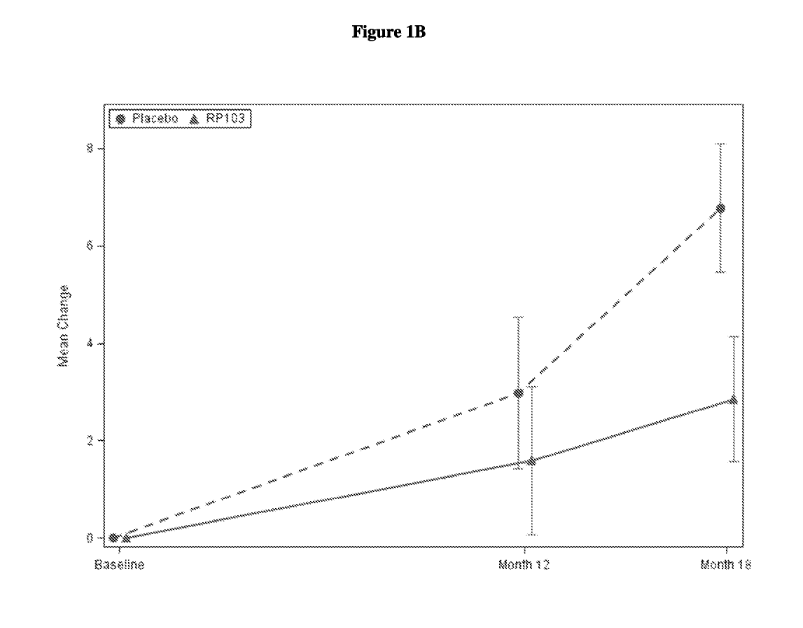

The ITT analysis of all 96 patients enrolled in the trial showed a positive trend toward slower progression of TMS, the primary endpoint of the study, in patients treated with RP103 versus those receiving placebo. The primary endpoint of change in TMS from baseline to 18 months was 6.68±7.98 in the placebo group and 4.55±8.24 in the RP103 group. The between group difference of 1.593±1.709 was not statistically significant when analyzed by the primary analysis method (95% CI [−5.000; 1.815]; p=0.3545). Supportive analysis showed a between group difference of 2.33±1.72 (95% CI [−5.750; 1.085]; p=0.1785) in the ITT population and 2.20±1.77 (95% CI [5.716; 1.324]; p=0.2181) in the PP population (Table 2). Though not statistically significant, reduction in TMS progression over 18 months due to RP103 treatment was 34% compared to those receiving placebo (4.5 versus 6.7 points change from baseline, respectively, p=0.19) (Table 2 and FIG. 1A). At baseline mean±sd. At 18 month mean change±se and treatment effect (RP103—placebo) from repeated measures mixed effect model with baseline, center, CAG repeats, age, and BMI as covariates.

TABLE 2

| | | | | |
|---|---|---|---|---|
| | | | 18-month | 18-month |
| | | TMS at | change from | treatment |
| Treatment | N | baseline | baseline | effect |

ITT population

| | | | | |
|---|---|---|---|---|
| Placebo | 45 | 24.9 ± 10.4 | 6.7 ± 1.2 | −2.2 ± 1.6 |
| RP103 | 51 | 23.5 ± 11.0 | 4.5 ± 1.1 | p = 0.19 |

NoTBZ

| | | | | |
|---|---|---|---|---|
| Placebo | 32 | 24.0 ± 9.8 | 6.8 ± 1.3 | −3.9 ± 1.8 |
| RP103 | 34 | 24.2 ± 12.1 | 2.8 ± 1.3 | p = 0.033 |

Among the 66 NoTBZ patients (32 on placebo and 34 on RP103), those receiving RP103 had a 57% slower progression in TMS compared to those receiving placebo (2.8 versus 6.5 points change from baseline, respectively, p=0.03) over 18 months (Table 2 and FIG. 1B).

Results showed a trend towards slower TMS progression in the ITT No TBZ subgroup (N=73) with a between group difference of −3.52±1.78 (95% CI [−7.067; 0.023]; p=0.0514). A statistically significant difference was found in the PP No TBZ subgroup (N=66) with a treatment effect on TMS of −3.69±1.74 (95% CI [−7.173; −0.210]; p=0.0381) which corresponds to a 57% reduction in TMS progression over 18 months due to treatment with RP103. Additional analysis was also performed on a subgroup of patients who were not treated with antipsychotics. The number of patients was small in each subgroup (46 patients in ITT No AP; 42 patients in PP No AP) and the difference between the two treatments was not of statistical significance in either of the subgroups.

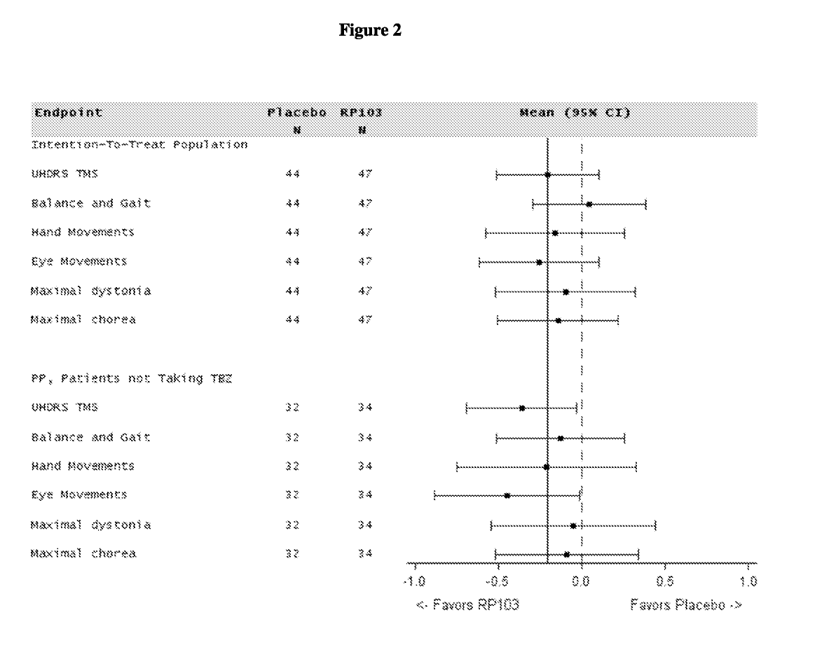

The slower progression of TMS in the NoTBZ resulted from improvements across multiple domains that comprise the UHDRS motor score: chorea subscore (1.0±0.5 for RP103 versus 1.6±0.6 for placebo, p=0.484), balance and gait subscore (0.3±0.2 for RP103 versus 0.5±0.2 for placebo, p=0.538), hand movements subscore (0.1±0.5 for RP103 versus 0.7±0.5 for placebo, p=0.329), and eye movements subscore (0.3±0.5 for RP103 versus 2.1±0.5 for placebo, p=0.016) (FIG. 2).

Effects on Secondary Endpoints

No significant effect of RP103 was observed on secondary endpoints in the ITT or NoTBZ population. The effect of RP103 on UHDRS motor subscales in the ITT population as well as the PP No TBZ subgroup was measured. A statistically significant difference in UHDRS motor subscales was observed for eye movements in the PP No TBZ population with a between group difference of −1.803±0.726 (95% CI [−3.253; −0.353]; p=0.0156).

Safety and Blood Concentrations of Cysteamine and BDNF

During the trial, 38 patients (86%) from the placebo group and 48 patients (92%) from the RP103 group experienced at least one adverse event. The most frequent adverse events were gastrointestinal complaints (61.5% of patients on RP103 and 45.5% of patients on placebo). Nine patients (17.3%) on RP103 and 3 patients (4.5%) on placebo complained of bad breath. Overall, 9 (10.4%) of the 96 enrolled patients experienced one or more serious adverse events (6 on RP103 and 4 on placebo). Five patients (5.2%) were discontinued from the study due to an adverse event (4 on RP103 [7.7%] and 1 on placebo [2.3%]).

As expected, all patients on placebo had a pre-dose cysteamine concentration under the limit of quantification. For the 49 patients (3 missing) from the RP103 group, the average Q12h pre-dose cysteamine concentration was 2.26±1.87 μmol/L. No statistically significant change in BDNF blood concentration was observed between the RP103 and placebo groups.

In this double-blind placebo controlled study, the ITT analysis including 96 patients showed a positive trend (p=0.19) toward slower progression of TMS in patients randomized to RP103 compared to those randomized to placebo. Despite the lack of statistical significance of the ITT analysis, this result is promising. A recent study reported an annual change of 3.73±0.26 (23). Indeed, the 18-month TMS change in the placebo group (6.7±1.2) is closer to the TMS change reported in this study rather than that of an older study, which involved fewer patients (22).

To evaluate the effect of RP103 without the possible confounding effects of tetrabenazine, patients not receiving tetrabenazine (NoTBZ) were analyzed separately for TMS and secondary endpoints in a subgroup analysis. It was found herein that RP103 was effective in slowing the deterioration in TMS compared with placebo (p=0.03). Taking into account that the number of patients not taking tetrabenazine is well balanced between treatment arms (32 on placebo and 34 on RP103) and between centers, it is believed that this result is clinically relevant. In addition, baseline TMS values were similar in RP103 and placebo arms in both ITT and NoTBZ populations and the TMS change under placebo was similar in both populations. RP103 effects in patients not receiving any antipsychotic drugs, which could also interfere with TMS evaluation (24), were also analyzed and it was observed that RP103 caused a 50% slower progression in TMS as compared to placebo (2.2 versus 4.4 points, respectively). However, this subgroup of patients was small (22 patients on placebo and 20 on RP103), not allowing statistical validation of the comparison. It is noteworthy that the effects of RP103 were less pronounced in the first 12 months and were most prominent after 18 months of treatment in both the ITT and NoTBZ populations (FIGS. 2A and 2B). This delay suggests that cysteamine could have a superimposed neuroprotective effect. In this study, however, no improvement was observed on functional and cognitive scales. This could be due to the lower sensitivity of these scales compared to TMS or to the fact that to cysteamine could act more efficiently on striatal cells than on cortical cells.

The first clinical trial of cysteamine in HD was conducted in 1986 (25) in five patients over 2 weeks. This trial showed no efficacy and was not followed by complementary studies. Thereafter, cystamine was tested in HD animal models mainly as an inhibitor of transglutaminase 2 (TG2) (9-13). TG2 is an enzyme, which is highly expressed in the central nervous system, that catalyzes the cross-linking and therefore the aggregation of proteins including the polyglutamine tracts of mutated huntingtin (26,27). TG2 activity is increased in brain cortex, striatum, cerebellum, and cortical nuclear extracts of patients affected with HD (28,29). Increase in TG2 activity is also deleterious independently of huntingtin aggregation by causing the formation of nuclear rods containing the cytoskeleton proteins actin and cofilin (30). In animal models, cystamine treatment decreased TG2 activity and showed promising efficacy on motor impairment, behavioral abnormalities and life expectancy. In addition, cystamine treatment reduced mutated huntingtin aggregates (10) and increased cell protection in the striatum as demonstrated by PET imaging (12).

Although all these experiments were performed using cystamine, it is likely that cysteamine is in fact responsible for the in vivo effects because cystamine is reduced intracellularly to cysteamine (15). Moreover, cysteamine crosses the blood-brain barrier whereas cystamine does not, or corsses less efficiently than cysteamine (31).

Multiple other effects beyond TG2 inhibition could explain the efficacy of cysteamine in HD. First, cysteamine inhibits the pro-apoptotic activity of caspase 3 independently of TG2 inhibition (32) and increases the production of heat shock proteins (9,33), which prevents protein misfolding and assists the refolding of misfolded proteins. Second, cysteamine may have a protective effect by increasing the level of antioxidants such as glutathione (14,32,34). Indeed, the mutant huntingtin protein causes mitochondrial dysfunctions resulting in an increase of oxidative damage mainly through the down-regulation of PGC-1α, the peroxisome proliferator-activated receptor gamma coactivator-1α, a key regulator of mitochondrial biogenesis and antioxidant defense (35). Third, cysteamine increases the release of BDNF, a crucial trophic factor that is involved in the survival of striatal neurons and is depleted in HD (14). Mechanisms by which cysteamine stimulates BDNF secretion involve increase of the heat shock DnaJ-containing protein 1b (HSJ1b) level and the inhibition of TG2 (14). Finally, cysteamine could be effective in HD by increasing the level of cysteine in the brain. Indeed, a recent study showed that neurodegeneration in HD was mediated by a transcriptional down-regulation of the gene coding cystathionine γ-lyase, resulting in a deficiency in this enzyme and ultimately in a deprivation in brain cysteine (36). This study also demonstrated that a cysteine-rich diet ameliorated motor abnormalities, partly reversed the brain and striatal atrophies and enhanced the survival in a mouse model of HD (36). Increased cysteine levels with cysteamine supplementation have been observed in both a cellular model and in brain of HD mouse model (11,37).

In conclusion, these results indicate that RP103 delayed-release formulation of cysteamine is safe and potentially effective in slowing the motor deterioration of HD. The effect of RP103 on TMS progression was significant on the subset of patients not treated with tetrabenazine. The open-label phase of the study is still ongoing and results at 36 months are expected to show the evolution of patients in the placebo group who continued treatment with RP103. Further studies involving a larger number of patients, as well as trials of RP103 in presymptomatic HTT mutation carriers can be carried out.

Numerous modifications and variations in the invention as set forth in the above illustrative examples are expected to occur to those skilled in the art. Consequently only such limitations as appear in the appended claims should be placed on the invention.

### REFERENCES

1. Ha A D, Fung V S C. Huntington's disease. Curr Opin Neurol 2012; 25(4):491-8.

2. The Huntington's Disease Collaborative Research Group. A novel gene containing a trinucleotide repeat that is expanded and unstable on Huntington's disease chromosomes. Cell 1993; 72(6):971-83.

3. Andrew S E, Paul Goldberg Y, Kremer B, et al. The relationship between trinucleotide (CAG) repeat length and clinical features of Huntington's disease. Nat Genet 1993; 4(4):398-403.

4. Zuccato C, Valenza M, Cattaneo E. Molecular mechanisms and potential therapeutical targets in Huntington's disease. Physiol Rev 2010; 90(3):905-81.

5. Labbadia J, Morimoto R I. Huntington's disease: underlying molecular mechanisms and emerging concepts. Trends Biochem Sci 2013; 38(8):378-85.

6. Roos RAC. Huntington's disease: a clinical review. Orphanet J Rare Dis 2010; 5(1):40.

7. Huntington Study Group. Tetrabenazine as antichorea therapy in Huntington disease: a randomized controlled trial. Neurology 2006; 66(3):366-72.

8. Pouladi M A, Morton A J, Hayden M R. Choosing an animal model for the study of Huntington's disease. Nat Rev Neurosci 2013; 14(10):708-21.

9. Karpuj M V, Becher M W, Springer J E, et al. Prolonged survival and decreased abnormal movements in transgenic model of Huntington disease, with administration of the transglutaminase inhibitor cystamine. Nat Med 2002; 8(2):143-9.

10. Dedeoglu A, Kubilus J K, Jeitner T M, et al. Therapeutic effects of cystamine in a murine model of Huntington's disease. J Neurosci 2002; 22(20):8942-50.

11. Fox J H, Barber D S, Singh B, et al. Cystamine increases L-cysteine levels in Huntington's disease transgenic mouse brain and in a PC12 model of polyglutamine aggregation. J Neurochem 2004; 91(2):413-22.

12. Wang X, Sarkar A, Cicchetti F, et al. Cerebral PET imaging and histological evidence of transglutaminase inhibitor cystamine induced neuroprotection in transgenic R6/2 mouse model of Huntington's disease. J Neurol Sci 2005; 231(1-2):57-66.

13. Van Raamsdonk J M, Pearson J, Bailey C D C, et al. Cystamine treatment is neuroprotective in the YAC128 mouse model of Huntington disease. J Neurochem 2005; 95(1):210-20.

14. Bone11-Pages M, Canals J M, Cordelières F P, et al. Cystamine and cysteamine increase brain levels of BDNF in Huntington disease via HSJ1b and transglutaminase. J Clin Invest 2006; 116(5):1410-24.

15. Jeitner T M, Delikatny E J, Ahlqvist J, Capper H, Cooper A J L. Mechanism for the inhibition of transglutaminase 2 by cystamine. Biochem Pharmacol 2005; 69(6):961-70.

16. Gahl W A, Thoene J G, Schneider J A. Cystinosis. N Engl J Med 2002; 347(2):111-21.

17. Gangoiti J A, Fidler M, Cabrera B L, Schneider J A, Barshop B A, Dohil R. Pharmacokinetics of enteric-coated cysteamine bitartrate in healthy adults: a pilot study. Br J Clin Pharmacol 2010; 70(3):376-82.

18. Huntington Study Group. Unified Huntington's Disease Rating Scale: reliability and consistency. Mov Disord 1996; 11(2):136-42.

19. Dubinsky R, Gray C. CYTE-I-HD: Phase I dose finding and tolerability study of cysteamine (Cystagon) in Huntington's disease. Mov Disord 2006; 21(4):530-3.

20. Langman C B, Greenbaum L A, Sarwal M, et al. A randomized controlled crossover trial with delayed-release cysteamine bitartrate in nephropathic cystinosis: effectiveness on white blood cell cystine levels and comparison of safety. Clin J Am Soc Nephrol 2012; 7(7):1112-20.

21. Dohil R, Cabrera B L, Gangoiti J A, Barshop B A, Rioux P. Pharmacokinetics of cysteamine bitartrate following intraduodenal delivery. Fundam Clin Pharmacol 2014; 28(2):136-43.

22. Bachoud-Lévi A C, Maison P, Bartolomeo P, et al. Retest effects and cognitive decline in longitudinal follow-up of patients with early HD. Neurology 2001; 56(8):1052-8.

23. Tabrizi S J, Scahill R I, Owen G, et al. Predictors of phenotypic progression and disease onset in premanifest and early-stage Huntington's disease in the TRACK-HD study: analysis of 36-month observational data. Lancet Neurol 2013; 12(7):637-49.

24. Désaméricq G, Dolbeau G, Verny C, et al. Effectiveness of anti-psychotics and related drugs in the Huntington French-speaking group cohort. PloS One 2014; 9(1):e85430.

25. Shults C, Steardo L, Barone P, et al. Huntington's disease: effect of cysteamine, a somatostatin-depleting agent. Neurology 1986; 36(8):1099-102.

26. Gentile V, Sepe C, Calvani M, et al. Tissue Transglutaminase-Catalyzed Formation of High-Molecular-Weight Aggregates in Vitro Is Favored with Long Polyglutamine Domains: A Possible Mechanism Contributing to CAG-Triplet Diseases. Arch Biochem Biophys 1998; 352(2):314-21.

27. Kahlem P, Green H, Djian P. Transglutaminase action imitates Huntington's disease: selective polymerization of Huntingtin containing expanded polyglutamine. Mol Cell 1998; 1(4):595-601.

28. Karpuj M V, Garren H, Slunt H, et al. Transglutaminase aggregates huntingtin into nonamyloidogenic polymers, and its enzymatic activity increases in Huntington's disease brain nuclei. Proc Natl Acad Sci 1999; 96(13):7388-93.

29. Lesort M, Chun W, Johnson G V, Ferrante R J. Tissue transglutaminase is increased in Huntington's disease brain. J Neurochem 1999; 73(5):2018-27.

30. Munsie L, Caron N, Atwal R S, et al. Mutant huntingtin causes defective actin remodeling during stress: defining a new role for transglutaminase 2 in neurodegenerative disease. Hum Mol Genet 2011; 20(10):1937-51.

31. Pinto J T, Khomenko T, Szabo S, et al. Measurement of sulfur-containing compounds involved in the metabolism and transport of cysteamine and cystamine. Regional differences in cerebral metabolism. J Chromatogr B Analyt Technol Biomed Life Sci 2009; 877(28):3434-41.

32. Lesort M, Lee M, Tucholski J, Johnson G V W. Cystamine inhibits caspase activity. Implications for the treatment of polyglutamine disorders. J Biol Chem 2003; 278(6):3825-30.

33. Chafekar S M, Duennwald M L. Impaired heat shock response in cells expressing full-length polyglutamine-expanded huntingtin. PloS One 2012; 7(5):e37929.

34. Kessler A, Biasibetti M, Feksa L R, et al. Effects of cysteamine on oxidative status in cerebral cortex of rats. Metab Brain Dis 2008; 23(1):81-93.

35. Johri A, Beal M F. Antioxidants in Huntington's disease. Biochim Biophys Acta 2012; 1822(5):664-74.

36. Paul B D, Sbodio J I, Xu R, et al. Cystathionine γ-lyase deficiency mediates neurodegeneration in Huntington's disease. Nature 2014; 509(7498):96-100.

37. Pinto J T, Van Raamsdonk J M, Leavitt B R, et al. Treatment of YAC128 mice and their wild-type littermates with cystamine does not lead to its accumulation in plasma or brain: implications for the treatment of Huntington disease. J Neurochem 2005; 94(4):1087-101.

What is claimed:

1. A method for treating Huntington's Disease comprising administering to a subject in need thereof a composition comprising cysteamine or a pharmaceutically acceptable salt thereof or cystamine or a pharmaceutically acceptable salt thereof in a total daily dose of approximately 1000 to 1500 mg given in two doses.

2. The method of claim 1 wherein the cysteamine or a pharmaceutically acceptable salt thereof or cystamine or a pharmaceutically acceptable salt thereof is administered in a total daily dose of approximately 1200 mg given in two doses.

3. The method of claim 2 wherein the administration is given in two daily doses of approximately 600 mg each.

4. The method of claim 1 or 2 wherein the cysteamine or a pharmaceutically acceptable salt thereof or cystamine or a pharmaceutically acceptable salt thereof is in a delayed release or extended release formulation.

5. The method of any one of claims 1-4, wherein the delayed release composition is enterically coated.

6. The method of claim 4 or 5, wherein the delayed release formulation comprises an enteric coating that releases the cysteamine or a pharmaceutically acceptable salt thereof or cystamine or a pharmaceutically acceptable salt thereof when the formulation reaches the small intestine or a region of the gastrointestinal tract of a subject in which the pH is greater than about pH 4.5.

7. The method of any one of claims 1-6, wherein the cysteamine or a pharmaceutically acceptable salt thereof or cystamine or a pharmaceutically acceptable salt thereof is formulated in a tablet or capsule which is enterically coated.

8. The method of any one of claims 1-7, wherein the administration results in a slower progression of total motor score compared to a subject not receiving cysteamine or a pharmaceutically acceptable salt thereof or cystamine or a pharmaceutically acceptable salt thereof.

9. The method of any one of claims 1-8, wherein the slower progression is a result in improvement in one or more motor scores selected from the group consisting of chorea subscore, balance and gait subscore, hand movements subscore, eye movement subscore and maximal dystonia subscore.

10. The method of any one of claims 1-9, further comprising administering to the subject an adjunct therapy.

11. The method of claim 10, wherein the adjunct therapy is selected of antipsychotics, antidepressants, vesicular monoamine transporter (VMAT)-inhibitors such as tetrabenazine, dopamine inhibitors, laquinimod, CNS-immunomodulators, neuroprotective factors, BDNF, ampakines, agents that upregulate BDNF, positive modulators of AMPA-type glutamate receptors, activators of BDNF receptor TrkB and gene therapy.

12. The method of any one of claims 1-10, wherein the subject is not concurrently taking tetrabenazine.

13. A method for slowing the progression of brain and striatal atrophies in a subject suffering from a neurodegenerative disease comprising administering to a subject in need thereof a composition comprising cysteamine or a pharmaceutically acceptable salt thereof or cystamine or a pharmaceutically acceptable salt thereof in a total daily dose of approximately 1000 to 1500 mg given in two doses.

14. A method for treating dystonia in a subject suffering from a neurodegenerative disease comprising administering to a subject in need thereof a composition comprising cysteamine or a pharmaceutically acceptable salt thereof or cystamine or a pharmaceutically acceptable salt thereof in a total daily dose of approximately 1000 to 1500 mg given in two doses.

15. The method of any one of claims 13-14, wherein the subject suffers from Huntington's disease.

16. The method of any one of the preceding claims, wherein the cysteamine or a pharmaceutically acceptable salt thereof or cystamine or a pharmaceutically acceptable salt thereof is administered parenterally.

17. The method of any one of the preceding claims, wherein the cysteamine or a pharmaceutically acceptable salt thereof or cystamine or a pharmaceutically acceptable salt thereof is administered orally.

18. The method of any one of the preceding claims, wherein the cysteamine or a pharmaceutically acceptable salt thereof or cystamine or a pharmaceutically acceptable salt thereof further comprises a pharmaceutically acceptable carrier.

19. The method of any one of the preceding claims, wherein the cysteamine or a pharmaceutically acceptable salt thereof or cystamine or a pharmaceutically acceptable salt thereof is formulated as a sterile pharmaceutical composition.

20. The method of any one of the preceding claims, wherein the method comprises administering cysteamine or a pharmaceutically acceptable salt thereof.

21. The method of claim 20, wherein the salt is cysteamine bitartrate.
